(19) United States
(12) Patent Application Publication
(10) Pub. No.: US 2010/0322926 A1
(43) Pub. Date: Dec. 23, 2010

(54) METHODS OF USING CADHERIN 11 (CDH11) ANTAGONISTS

(75) Inventor: Pierre Saint-Mezard, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG, Basel (CH)

(21) Appl. No.: 12/866,796

(22) PCT Filed: Feb. 9, 2009
(86) PCT No.: PCT/EP2009/051467

§ 371 Date: Aug. 9, 2010

Related U.S. Application Data
(60) Provisional application No. 61/065,381, filed on Feb. 11, 2008.

(51) Int. Cl. A61K 39/395; C12Q 1/68; G01N 33/53; A61K 38/00; A61K 31/7088

(52) U.S. Cl. 424/133.1; 424/130.1; 424/178.1; 435/6; 435/7.1; 514/1.1; 514/44 R; 514/44 A

(57) ABSTRACT

The present invention provides novel methods of inhibiting or preventing epithelial-mesenchymal transition (EMT) or endothelial-mesenchymal transition (EnMT), such as EMT or EnMT, associated with fibrosis and chronic tissue rejection. The present invention also provides methods for diagnosing or assessing whether a subject has or is at risk of developing a disease associated with CDH11 expression, as well as methods for determining the prognosis of a subject diagnosed with a CDH11-associated condition. The invention employs CDH11 antagonists to downmodulate CDH11 activity, thereby inhibiting EMT or EnMT.

### BACKGROUND OF THE INVENTION

During embryonic development, epithelial and mesenchymal phenotypic transitions are necessary for the correct formation of different tissues. However, these processes are not only involved in development but they are also thought to be implicated in pathological conditions such as cancer and fibrosis (Kalluri, R. and Neilson, E. G., J. Clin. Invest. 112, 1776-1784 (2003)). Epithelial-Mesenchymal Transition (hereinafter “EMT”) is a program whereby injured epithelial cells that function as ion and fluid transporters become matrix remodeling mesenchymal cells. This process requires either transcriptional repression of genes that maintain the epithelial phenotype and transcriptional activation, or relieved repression of genes needed for functional myofibroblasts. One striking example of EMT is observed in kidney development and disease. During development, mesenchymal cells are initially formed by EMT and subsequently, some of these cells undergo mesenchymal to epithelial transition (MET) to form the epithelia of the pronephros, mesonephros and metanephros (Dressler, G. R., Trends in Cell Biology 12, 390-395 (2002)). In the adult, EMT is implicated in the development of end-stage kidney failure in most progressive renal diseases (Liu, Y., J. Am. Soc. Nephrol., 15, 1-12 (2004)). Thus, while kidney formation involves reciprocal transformations, suppression of this plasticity in the adult is critical to maintain normal tissue architecture and homeostasis.

### SUMMARY OF THE INVENTION

The present invention provides novel methods of inhibiting or preventing epithelial-mesenchymal transition (EMT) or endothelial-mesenchymal transition (EnMT) associated with fibrosis in a subject, by administering a therapeutically effective amount of a CDH11 antagonist.

The present invention also provides novel methods of treating particular diseases or conditions associated with CDH11 activity, including, but not limited to, vascular fibrosis, pulmonary hypertension, kidney fibrosis, nephronopthisis, liver fibrosis, skin fibrosis, lung fibrosis, fibrosis of the joint (e.g., rheumatoid arthritis), fibrosis of the mesothelium and fibrosis of the gut (e.g., inflammatory bowel diseases). In a particular embodiment, the methods of the present invention are used to treat kidney fibrosis.

In another embodiment, the methods of the present invention can be used to prevent or reduce the severity of chronic tissue rejection (i.e., of a transplanted or grafted tissue) in a subject. Exemplary tissues include, but are not limited to, whole blood, blood vessels, bones, corneas, as well as major organs such as hearts, kidneys, livers, eyes, lungs, and pancreases.

The present invention further provides methods of assessing whether a subject has or is at risk of developing a CDH11-associated condition, such as EMT, EnMT, fibrosis, and chronic tissue rejection, by assaying a sample for CDH11 and interpreting an aberrant or elevated level of CDH11 as indicating the subject has or is at risk of developing a CDH11-associated condition.

Also provided are methods for diagnosing a CDH11-associated condition, such as EMT, EnMT, fibrosis, and chronic tissue rejection, by contacting a target sample with a reagent (e.g., a detectably labeled antibody or nucleic acid) which reacts with CDH11, detecting CDH11 and interpreting an elevated concentration of CDH11 relative to a normal control as being indicative of a CDH11-associated condition.

The present invention also provides methods of determining the prognosis of a subject diagnosed with a CDH11-associated condition (e.g., EMT, EnMT, fibrosis, and chronic tissue rejection) by assaying at least two samples from the subject which have been collected over time, comparing the levels of CDH11 in each sample, and determining if the level of CDH11 has increased or decreased over time, wherein an increase in CDH11 is indicative of an increase in severity of the condition and a decrease in CDH11 is indicative of a decrease in severity of the condition. In a particular embodiment, the prognosis of a patient who has been treated with a CDH11 antagonist is determined.

A wide variety of CDH11 antagonists can be used in the methods of the present invention, such as antibodies, fusion proteins, nucleic acids (e.g., antisense molecules, such as RNA interfering agents and ribozymes), immunoconjugates (e.g., an antibody linked to a therapeutic agent, such as a cytotoxic agent, immunosuppressive agent or a chemotherapeutic agent), small molecules, fusion proteins, and CDH11-derived peptidic compounds.

In a particular embodiment, the CDH11 antagonist is an antibody (of fragment thereof). Antibodies suitable for protection according to the invention include all known forms of antibodies having at least variable region sequences. For example, the antibody can be a murine, human, humanized, chimeric or bispecific monoclonal antibody. The antibody can be a Fab, Fab′2, ScFv, SMIP, affibody, avimer, nanobody, and a domain antibody and the antibody can be an IgG1, IgG2, IgG3, IgG4, IgM, IgA1, IgA2, IgAsec, IgD, or IgE antibody.

CDH11 antagonists utilized in the methods of the present invention can be administered alone or in combination with other therapeutic agents. For example, the antibodies can be administered in combination with (i.e., together with or linked to) cytotoxins, other known therapeutic agents (i.e., immunosuppressive or chemotherapeutic agents; and/or other therapeutic antibodies. In one embodiment, the antagonist is linked to a second binding molecule, such as an antibody (i.e., thereby forming a bispecific molecule) or other binding agent that binds to a different target or a different epitope on CDH11.

Other features and advantages of the invention will be apparent from the following detailed description, and from the claims.

### BRIEF DESCRIPTION OF THE DRAWINGS

FIG. 1 is a schematic representation depicting the overlap between genes associated with chronic allograft rejection and genes correlating with Snail2, an EMT marker that is highly up-regulated in both early and late chronic rejection.

FIG. 2 is a gene set enrichment method (GSEA) representation of the EMT set for (A) both progressor and non-progressor patients from the Hannover data set and (B) control and chronic allograft rejection Grade III patient samples.

FIG. 3 shows the mean mRNA CDH11 expression for (A) protocol biopsies of kidneys from non-progressor and progressor patents three months post-kidney transplant and (B) diagnostic biopsies for control, borderline, Grade I, Grade II and Grade III kidney transplanted patients.

FIG. 4 is a photographic depiction of the immunohistochemistry of CDH11 on paraffin embedded kidney cortex biopsies of healthy and transplanted patients.

FIG. 5 depicts CDH11 mRNA expression levels in acute and chronic rejection samples from kidney transplanted cynomolgus monkeys.

FIG. 6 is a photographic depiction of the immunohistochemistry of CDH11 in frozen biopsies from heart transplanted mice.

FIG. 7 depicts CDH11 mRNA expression levels in tissues from unilateral ureteral obstruction (UUO) mouse models, demonstrating that CDH11 is an EMT marker in fibrotic disorders.

### DETAILED DESCRIPTION OF THE INVENTION

In order that the present invention may be more readily understood, certain terms are first defined. Additional definitions are set forth throughout the detailed description.

I. Definitions

As used herein, the term “cadherins” refers to a family of Ca2+-dependent cell-cell adhesion molecules. All cadherins are single-pass transmembrane proteins with a variable number of 110 amino acid extracellular cadherin (EC) domains. Classical cadherins contain five EC domains and a conserved cytoplasmic domain. Based on amino acid alignment, classical cadherins are divided into type I and type II subgroups. Type I cadherins, including cadherins E (epithelial), N (neural), P (placental), and R (retinal) cadherin differ from type II cadherins in their specific amino acid sequences. Type II cadherins include human cadherin-5, -6, -8, -11, and -12.

As used herein, the term “Cadherin 11” (also referenced interchangeably herein as “CDH11,” “Cadherin 11, type 2, ON-cadherin (osteoblast),” “CAD11,” “CDHOB,” “OB-Cadherin,” and “OSF-4”) refers to a member of the cadherin family that is a marker of the loosely connected and migratory cellular elements of the mesenchyme. Strong expression of CDH11 has been noted in brain, spinal cord, bone marrow and bone cells, and is regulated in human endometrial glandular epithelial and stromal cells. During development, CDH11 expression is associated with mesenchymal morphogenesis in the head, somite, and limb bud of early mouse embryos and it is also strongly expressed in mesenchyme during lung or kidney branching morphogenesis. Whereas epithelial cadherins, such as E-cadherin, are responsible for the formation and maintenance of epithelial structures, expression of CDH11 correlates with a migratory cellular phenotype and is a critical determinant for cell motility, cell intercalation, tissue extension and myofibroblast differentiation, as taught for example, by Borchers, A., et al., Development, 128, 3049-3060 (2001); Desmouliere, A., et al., J. Cell Biol., 122, 103-111 (1993); Hinz, B., et al., Mol. Biol. Cell, 15, 4310-4320 (2004); Kiener, H. P et al., Mol. Biol. Cell, 17, 2366-2376 (2006); Kimura, Y. et al., Developmental Biology, 169, 347-358 (1995); Locascio, A. et al., Current Opinion in Genetics & Development, 11, 464-469 (2001); Okazaki, M. et al., J. Biol. Chem., 269, 12092-12098 (1994); Pishvaian, M. J. et al., Cancer Res., 59, 947-952 (1999); Shibata, T., et al., Cancer Letters, 99, 147-153 (1996); Tomita, K. et al., Cancer Res., 60, 3650-3654 (2000); and Valencia, X. et al., J. Exp. Med., 200, 1673-1679 (2004), the contents of which are expressly incorporated herein by reference.

Representative CDH11 sequences, include, but are not limited to, the sequences set forth below.

Homo sapiens cadherin 11, type 2, OB-cadherin
(osteoblast) (CDH11) (NM_001797)
(SEQ ID NO: 1)
MKENYCLQAALVCLGMLCHSHAFAPERRGHLRPSFHGHHEKGKEGQVLQR
SKRGWVWNQFFVIEEYTGPDPVLVGRLHSDIDSGDGNIKYILSGEGAGTI
FVIDDKSGNIHATKTLDREERAQYTLMAQAVDRDTNRPLEPPSEFIVKVQ
DINDNPPEFLHETYHANVPERSNVGTSVIQVTASDADDPTYGNSAKLVYS
ILEGQPYFSVEAQTGIIRTALPNMDREAKEEYHVVIQAKDMGGHMGGLSG
TTKVTITLTDVNDNPPKFPQSVYQMSVSEAAVPGEEVGRVKAKDPDIGEN
GLVTYNIVDGDGMESFEITTDYETQEGVIKLKKPVDFETKRAYSLKVEAA
NVHIDPKFISNGPFKDTVTVKISVEDADEPPMFLAPSYIHEVQENAAAGT
VVGRVHAKDPDAANSPIRYSIDRHTDLDRFFTINPEDGFIKTTKPLDREE
TAWLNITVFAAEIHNRHQEAKVPVAIRVLDVNDNAPKFAAPYEGFICESD
QTKPLSNQPIVTISADDKDDTANGPRFIFSLPPEIIHNPNFTVRDNRDNT
AGVYARRGGFSRQKQDLYLLPIVISDGGIPPMSSTNTLTIKVCGCDVNGA
LLSCNAEAYILNAGLSTGALIAILACIVILLVIVVLFVTLRRQKKEPLIV
FEEEDVRENIITYDDEGGGEEDTEAFDIATLQNPDGINGFIPRKDIKPEY
QYMPRPGLRPAPNSVDVDDFINTRIQEADNDPTAPPYDSIQIYGYEGRGS
VAGSLSSLESATTDSDLDYDYLQNWGPRFKKLADLYGSKDTFDDDS

As used herein, the term “CDH11 antagonist” refers to any agent which downmodulates CDH11 activity, including agents which downregulate CDH11 expression or inhibit CDH11 function (e.g., its ability to induce cell migration). Such inhibitory agents can, for example, inhibit or block CDH11-mediated cellular interaction.

As used herein, the term “down-modulates” refers to any statistically significant decrease in the biological activity of CDH11, including full blocking of the activity (i.e., inhibition). For example, “down-modulation” can refer to a decrease of about 10%, 20%, 30%, 40%, 50%, 60%, 70%, 80%, 90%, or 100% in CDH11 expression.

As used herein, the term “Epithelial-Mesenchymal Transition” (EMT) refers to the conversion from an epithelial to a mesenchymal phenotype, which is a normal process of embryonic development. EMT is also the process whereby injured epithelial cells that function as ion and fluid transporters become matrix remodeling mesenchymal cells. In carcinomas, this transformation results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility (See Vincent-Salomon et al., Breast Cancer Res. 2003; 5(2): 101-106). Classes of molecules that change in expression, distribution, and/or function during EMT, and that are causally involved, include growth factors (e.g., transforming growth factor (TGF)-β, wnts), transcription factors (e.g., snails, SMAD, LEF, and nuclear β-catenin), molecules of the cell-to-cell adhesion axis (cadherins, catenins), cytoskeletal modulators (Rho family), and extracellular proteases (matrix metalloproteinases, plasminogen activators) (see Thompson et al., Cancer Research 65, 5991-5995, Jul. 15, 2005).

As used herein, the term “Mesenchymal-to-Epithelial Transition” (MET) refers to a fundamental embryologic process, whereby mesenchymal cells transition into epithelial cells to form the epithelia of the pronephros, mesonephros and metanephros. Several growth factor families are critical regulators of kidney MET, including the wnt/wingless and bone morphogenetic protein families. In particular, fibroblast growth factors (FGF), FGF receptors (FGFR), and proteoglycans that modulate FGF signaling are essential modulators of nephrogenic MET (see Chaffer et al., Cancer Research 66, 11271-11278, Dec. 1, 2006).

As used herein, the term “Endothelial Mesenchymal Transition” (EnMT) refers to the phenotypic conversion of endothelial cells to a mesenchymal-myofibroblast phenotype.

As used herein, the term “epithelium” refers to the covering of internal and external surfaces of the body, including the lining of vessels and other small cavities. It consists of a collection of epithelial cells forming a relatively thin sheet or layer due to the constituent cells being mutually and extensively adherent laterally by cell-to-cell junctions. The layer is polarized and has apical and basal sides. Despite the tight regimentation of the epithelial cells the epithelium does have some plasticity and cells in an epithelial layer can alter shape, such as change from flat to columnar, or pinch in at one end and expand at the other. However, these tend to occur in cell groups rather than individually (see Thompson et al., Cancer Research 65, 5991-5995, Jul. 15, 2005).

As used herein, the term “mesenchyme” refers to the part of the embryonic mesoderm, consisting of loosely packed, unspecialized cells set in a gelatinous ground substance, from which connective tissue, bone, cartilage, and the circulatory and lymphatic systems develop. Mesenchyme is a collection of cells which form a relatively diffuse tissue network. Mesenchyme is not a complete cellular layer and the cells typically have only points on their surface engaged in adhesion to their neighbors. These adhesions may also involve cadherin associations (see Thompson et al., Cancer Research 65, 5991-5995, Jul. 15, 2005).

As used herein, the term “fibrosis” refers to the formation or development of excess fibrous connective tissue in an organ or tissue as a reparative or reactive process, as opposed to a formation of fibrous tissue as a normal constituent of an organ or tissue. Examples of fibrosis include, but are not limited to vascular fibrosis, vascular fibrosis associated with pulmonary hypertension, kidney fibrosis, liver fibrosis, skin fibrosis, lung fibrosis, fibrosis of the joint (e.g., rheumatoid arthritis), fibrosis of the mesothelium, fibrosis of the eyes, and fibrosis of the gut (e.g., inflammatory bowel diseases).

As used herein, the term “interstitial fibrosis” refers to fibrosis relating to or situated in the small, narrow spaces between tissues or parts of an organ. For example, interstitial pulmonary fibrosis (also known as interstitial lung disease and pulmonary fibrosis) refers to fibrosis (i.e., scarring) of the interstitium, i.e., the tissue between the air sacs of the lungs. Additionally, renal interstitial fibrosis (also known as kidney fibrosis) is characterized by the destruction of renal tubules and interstitial capillaries as well as by the accumulation of extracellular matrix proteins.

As used herein, the term “vascular remodeling” is a type of fibrosis that refers to the active process of structural and cellular changes in the vasculature. All of these changes are characterized by an increased number of cells which express alpha-smooth muscle actin. This accumulation of alpha-smooth muscle positive cells could result from the proliferative expansion of resident vascular smooth muscle cells (SMC), recruitment of circulating progenitor cells to sites of vascular injury, or transition of endothelial cells towards a mesenchymal phenotype (EnMT).

As used herein, the term “transplantation” refers to the process of taking a cell, tissue, or organ, called a “transplant” or “graft” from one subject and placing it into a (usually) different subject. The subject who provides the transplant is called the “donor” and the subject who received the transplant is called the “recipient.” An organ, or graft, transplanted between two genetically different subjects of the same species is called an “allograft”. A graft transplanted between subjects of different species is called a “xenograft”.

As used herein, the term “transplant rejection” is defined as functional and structural deterioration of the organ due to an active immune response expressed by the transplant recipient, and independent of non-immunologic causes of organ dysfunction.

As used herein, the term “acute rejection” (e.g., of a transplant) refers to a rejection of a transplanted organ developing after the first 5-60 post-transplant days. It is generally a manifestation of cell-mediated immune injury. It is believed that both delayed hypersensitivity and cytotoxicity mechanisms are involved. The immune injury is directed against HLA, and possibly other cell-specific antigens expressed by the tubular epithelium and vascular endothelium.

As used herein, the term “chronic rejection” (e.g., of a transplant) represents a consequence of combined immunological injury (e.g. chronic rejection) and non-immunological damage (e.g. hypertensive nephrosclerosis, or nephrotoxicity of immuno-suppressants like cyclosporine A), occurring months or years after transplantation and ultimately leading to fibrosis and sclerosis of the allograft, associated with progressive loss of organ function.

The most common histologic manifestation is a progressive narrowing of the muscular arteries, which is often referred to as “graft vascular disease” or “obliterative arteriopathy (OA)”, and can be likened to an accelerated form of atherosclerosis. Obliterative arteriopathy damages the allograft primarily by compromising the arterial blood flow, predisposing it to chronic ischemic damage and infarction. Other common characteristics of chronic allograft rejection include patchy interstitial inflammation, fibrosis and associated parenchymal atrophy, destruction of epithelial-lined conduits such as bronchioles in lung allografts and bile ducts in the liver, and depletion of organ-associated lymphoid tissue.

As used herein, the terms “Grade I rejection” or “Grade I allograft rejection” refers to mild interstitial fibrosis and tubular atrophy (<25% of the cortex).

As used herein, the terms “Grade II rejection” or “Grade II allograft rejection” refers to moderate interstitial fibrosis and tubular atrophy (between 25 and 50%_of the cortex.

As used herein, the terms “Grade III rejection” or “Grade III allograft rejection” refers to severe interstitial fibrosis and tubular atrophy (>50% of the cortex).

As used herein, the terms “progressor” or “progressor patient” refers to the recipient of a transplant who will develop chronic rejection in the next 6 to 12 month.

As used herein, the terms “non-progressor”, “stable”, “stable patient,” or “non-progressor patient” refers to the recipient of a transplant who will display stable graft function in the next 6 to 12 month.

As used herein, the term “intimal hyperplasia” refers to the universal response of a vessel to injury. It involves the coordinated stimulation of smooth muscle cells by mechanical, cellular and humoral factors to induce a program of cellular activation that leads to proliferation, migration and extracellular matrix deposition. Intimal hyperplasia can cause late bypass graft failure, particularly in vein and synthetic vascular grafts.

II. CDH11 Antagonists

As used herein, the term “antagonist” refers to any agent which downmodulates CDH11 activity, including agents which downregulate CDH11 expression or inhibit CDH11 function (e.g., its ability to induce cell migration). Such inhibitory agents can, for example, inhibit or block CDH11-mediated cellular interaction. Representative antagonists, include, but are not limited to antibodies, nucleic acids (e.g., antisense molecules, such as ribozymes and RNA interfering agents), immunoconjugates (e.g., an antibody linked to a therapeutic agent, such as a cytotoxic agent, an immunosuppressive agent or a chemotherapeutic agent), small molecule inhibitors, fusion proteins, and CDH11-derived peptidic compounds.

A. Antibodies

In one embodiment of the invention, the invention employs an antibody that binds CDH11 and inhibits CDH11 activity and/or down-modulates CDH11 expression. For example, the antibody can bind to CDH11 and interfere with CDH11-mediated cellular interaction. The term “antibody” or “immunoglobulin,” as used interchangeably herein, includes whole antibodies and any antigen binding fragment (i.e., “antigen-binding portion”) or single chains thereof. An “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., CDH11). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al. (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242, 423-426; and Huston et al. (1988) Proc. Natl. Acad. Sci. USA 85, 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique and those described herein such as, for example, a hybridoma method, as described by Kohler et al. (1975) Nature, 256:495, a transgenic animal, as described by, for example, (see e.g., Lonberg, et al. (1994) Nature 368(6474): 856-859), recombinant DNA methods (see, e.g., U.S. Pat. No. 4,816,567), or using phage antibody libraries using the techniques described in, for example, Clackson et al., Nature, 352:624-628 (1991) and Marks et al., J. Mol. Biol., 222:581-597 (1991). Monoclonal antibodies include chimeric antibodies, human antibodies and humanized antibodies and may occur naturally or be recombinantly produced.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “human antibody,” as used herein, is intended to include antibodies having variable regions in which both the framework and CDR regions are derived from human germline immunoglobulin sequences as described, for example, by Kabat et al. (See Kabat, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue which is not encoded by the human germline immunoglobulin sequence. Typically, the human antibody can have up to twenty positions replaced with amino acid residues which are not part of the human germline immunoglobulin sequence. In a particular embodiment, these replacements are within the CDR regions as described in detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79, 315-321; Kostelny et al. (1992) J. Immunol. 148, 1547-1553.

As used herein, a “heterologous antibody” is defined in relation to the transgenic non-human organism or plant producing such an antibody.

An “isolated antibody,” as used herein, is intended to refer to an antibody which is substantially free of other antibodies having different antigenic specificities (e.g., an isolated antibody that specifically binds to CDH11 is substantially free of antibodies that specifically bind antigens other than CDH11). In addition, an isolated antibody is typically substantially free of other cellular material and/or chemicals. In one embodiment of the invention, a combination of “isolated” monoclonal antibodies having different CDH11 binding specificities are combined in a well defined composition.

As used herein, “isotype” refers to the antibody class (e.g., IgM or IgG1) that is encoded by heavy chain constant region genes. In one embodiment, an antibody or antigen binding portion thereof is of an isotype selected from an IgG1, an IgG2, an IgG3, an IgG4, an IgM, an IgA1, an IgA2, an IgAsec, an IgD, or an IgE antibody isotype.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence regions in a gene encoding an antibody. Non-classical isotype switching may occur by, for example, homologous recombination between human σ_{μ} and human Σ_{μ} (δ-associated deletion). Alternative non-classical switching mechanisms, such as intertransgene and/or interchromosomal recombination, among others, may occur and effectuate isotype switching.

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See, for example, PCT Publication WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for CDH11. In certain embodiments herein, the camelid antibody or nanobody is naturally produced in the camelid animal, i.e., is produced by the camelid following immunization with CDH11 or a peptide fragment thereof, using techniques described herein for other antibodies. Alternatively, the anti-CDH11 camelid nanobody is engineered, i.e., produced by selection, for example from a library of phage displaying appropriately mutagenized camelid nanobody proteins using panning procedures with CDH11 or a CDH11 epitope described herein as a target. Engineered nanobodies can further be customized by genetic engineering to increase the half life in a recipient subject from 45 minutes to two weeks.

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}⁻V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21).

A diabody can be fused to Fc to generate a “di-diabody” (see Lu et al., 2004 J. Biol. Chem., 279(4):2856-65).

The invention further provides CDH11 binding molecules that exhibit functional properties of antibodies but derive their framework and antigen binding portions from other polypeptides (e.g., polypeptides other than those encoded by antibody genes or generated by the recombination of antibody genes in vivo). The antigen binding domains (e.g., CDH11 binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., hCDH11) and for other functions (e.g., inhibition of biological activity of CDH11). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

Antibodies that can be used in the methods of the present invention also include those antibodies that bind the same or an overlapping epitope as the particular antibodies described herein, i.e., antibodies that compete for binding to CDH11, or bind to an epitope on CDH11 recognized by the particular antibodies described herein.

Antibodies that recognize the same or an overlapping epitope can be identified using routine techniques such as an immunoassay, for example, by showing the ability of one antibody to block the binding of another antibody to a target antigen, i.e., a competitive binding assay. Competitive binding is determined in an assay in which the immunoglobulin under test inhibits specific binding of a reference antibody to an antigen, such as CDH11. Numerous types of competitive binding assays are known, for example: solid phase direct or indirect radioimmunoassay (RIA), solid phase direct or indirect enzyme immunoassay (EIA), sandwich competition assay (see Stahli et al., (1983) Methods in Enzymology 9:242); solid phase direct biotin-avidin EIA (see Kirkland et al., (1986) J. Immunol. 137:3614); solid phase direct labeled assay, solid phase direct labeled sandwich assay (see Harlow and Lane, (1988) Antibodies: A Laboratory Manual, Cold Spring Harbor Press); solid phase direct label RIA using I-125 label (see Morel et al., (1988) Mol. Immunol. 25(1):7); solid phase direct biotin-avidin EIA (Cheung et al., (1990) Virology 176:546); and direct labeled RIA. (Moldenhauer et al., (1990) Scand. J. Immunol. 32:77). Typically, such an assay involves the use of purified antigen (e.g., CDH11) bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

As used herein, the terms “specific binding,” “specifically binds,” “selective binding,” and “selectively binds,” mean that an antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). Specific or selective binding can be determined according to any art-recognized means for determining such binding, including, for example, according to Scatchard analysis and/or competitive binding assays.

The term “K_{D},” as used herein, is intended to refer to the dissociation equilibrium constant of a particular antibody-antigen interaction or the affinity of an antibody for an antigen. In one embodiment, the antibody or antigen binding portion thereof according to the present invention binds an antigen (e.g., CDH11) with an affinity (K_{D}) of 50 nM or better (i.e., or less) (e.g., 40 nM or 30 nM or 20 nM or 10 nM or less), as measured using a surface plasmon resonance assay or a cell binding assay. In a particular embodiment, an antibody or antigen binding portion thereof according to the present invention binds CDH11 with an affinity (K_{D}) of 8 nM or better (e.g., 7 nM, 6 nM, 5 nM, 4 nM, 2 nM, 1.5 nM, 1.4 nM, 1.3 nM, 1nM or less), as measured by a surface plasmon resonance assay or a cell binding assay. In other embodiments, an antibody or antigen binding portion thereof binds an antigen (e.g., CDH11) with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument using recombinant CDH11 as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen.

The term “K_{off},” as used herein, is intended to refer to the off rate constant for the dissociation of an antibody from the antibody/antigen complex.

The term “EC50,” as used herein, refers to the concentration of an antibody or an antigen-binding portion thereof, which induces a response, either in an in vitro or an in vivo assay, which is 50% of the maximal response, i.e., halfway between the maximal response and the baseline.

As used herein, “glycosylation pattern” is defined as the pattern of carbohydrate units that are covalently attached to a protein, more specifically to an immunoglobulin protein.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “modifying,” or “modification,” as used herein, is intended to refer to changing one or more amino acids in the antibodies. The change can be produced by adding, substituting or deleting an amino acid at one or more positions. The change can be produced using known techniques, such as PCR mutagenesis. For example, in some embodiments, an antibody employed by the methods of the present invention can be modified, to thereby modify the binding affinity of the antibody to CDH11.

The present invention also encompasses “conservative amino acid substitutions” in the sequences of the antibodies used in the methods of the invention, i.e., nucleotide and amino acid sequence modifications which do not abrogate the binding of the antibody encoded by the nucleotide sequence or containing the amino acid sequence, to the antigen, i.e., CDH11. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-CDH11 antibody of the present invention is preferably replaced with another amino acid residue from the same class. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:.412-417 (1997)).

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-CDH11 antibody coding sequence, such as by saturation mutagenesis, and the resulting modified anti-CDH11 antibodies can be screened for binding activity.

A “consensus sequence” is a sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence.

Similarly, the consensus sequence for the CDRs of can be derived by optimal alignment of the CDR amino acid sequences of CDH11 antibodies of the present invention.

### Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains Appropriate scaffolds form a conformationally stable framework that displays the grafted residues such that they form a localized surface and bind the target of interest (e.g., CDH11). For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγR1, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of a CDH11 binding polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The polypeptides containing these novel amino acids can then be pegylated at these chosen sites in the protein.

B. Immunoconjugates

In another aspect, the present invention employs immunoconjugate agents that target CDH11 and which inhibit or down-modulate CDH11. Agents that can be targeted to CDH11 include, but are not limited to, chemotherapeutic agents, cytotoxic agents, anti-inflammatory agents, e.g., a steroidal or nonsteroidal inflammatory agent, or a cytotoxin antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The term “cytotoxin” or “cytotoxic agent” includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Immunoconjugates can be formed by conjugating (e.g., chemically linking or recombinantly expressing) antibodies to suitable therapeutic agents. Suitable agents include, for example, a cytotoxic agent, a toxin (e.g. an enzymatically active toxin of bacterial, fungal, plant or animal origin, or fragments thereof), and/or a radioactive isotope (i.e., a radioconjugate). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated anti-CDH11 antibodies. Examples include ²¹²Bi, ¹³¹I, ¹³¹In, ₉₀Y and ¹⁸⁶Re.

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., WO94/11026).

C. Small Molecule Inhibitors

In another embodiment, the CDH11 antagonist employed in the invention is a small molecule inhibitor. As used herein, the term “small molecule inhibitor” is a term of the art and includes molecules that are less than about 7500, less than about 5000, less than about 1000 molecular weight or less than about 500 molecular weight, and inhibit CDH11 activity. Exemplary small molecule inhibitors include, but are not limited to, peptides, peptidomimetics, nucleic acids, carbohydrates, small organic molecules (e.g., Cane et al. 1998. Science 282:63), and natural product extract libraries. In another embodiment, the compounds are small, organic non-peptidic compounds. Like antibodies, these small molecule inhibitors can bind to and/or otherwise block CDH11-mediated cellular interaction.

D. Nucleic Acids/Antisense Molecules

In another embodiment, the CDH11 antagonist employed in the present invention is an antisense nucleic acid molecule that is complementary to a gene encoding CDH11, or to a portion of said gene, or a recombinant expression vector encoding said antisense nucleic acid molecule. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

The use of antisense nucleic acids to down-modulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Antisense nucleic acids can be designed according to the rules of Watson and Crick base pairing. The antisense nucleic acid molecule can be complementary to the entire coding region of CDH11 mRNA, but more preferably is an oligonucleotide which is antisense to only a portion of the coding or noncoding region of CDH11 mRNA. For example, the antisense oligonucleotide can be complementary to the region surrounding the translation start site of CDH11 mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules that can be utilized in the methods of the present invention are typically administered to a subject or generated in situ such that they hybridize with or bind to cellular mRNA and/or genomic DNA encoding CDH11 to thereby inhibit expression of the CDH11, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule employed by the methods of the present invention can include an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid used in the methods of the present invention is a compound that mediates RNAi. RNA interfering agents include, but are not limited to, nucleic acid molecules including RNA molecules which are homologous to the CDH11 or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

In still another embodiment, an antisense nucleic acid is a ribozyme. Ribozymes are catalytic RNA molecules with ribonuclease activity which are capable of cleaving a single-stranded nucleic acid, such as an mRNA, to which they have a complementary region. Thus, ribozymes (e g., hammerhead ribozymes (described in Haselhoff and Gerlach, 1988, Nature 334:585-591) can be used to catalytically cleave CDH11 mRNA transcripts to thereby inhibit translation of CDH11 mRNA.

Alternatively, gene expression can be inhibited by targeting nucleotide sequences complementary to the regulatory region of CDH11 (e.g., the CDH11 promoter and/or enhancers) to form triple helical structures that prevent transcription of the CDH11 gene in target cells. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12):807-15.

E. Fusion Proteins and CDH11-Derived Peptidic Compounds

In another embodiment, the CDH11 antagonist used in the methods of the present invention is a fusion protein or peptidic compound derived from the CDH11 amino acid sequence. In particular, the inhibitory compound comprises a fusion protein or a portion of CDH11 (or a mimetic thereof) that mediates interaction of CDH11 with a target molecule such that contact of CDH11 with this fusion protein or peptidic compound competitively inhibits the interaction of CDH11 with the target molecule. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the CDH11 fusion protein or peptidic compounds of the invention can be improved by making peptide modifications, such as the addition of N-linked glycosylation sites into CDH11, or conjugating CDH11 to poly(ethylene glycol) (PEG; pegylation), e.g., via lysine-monopegylation. Said techniques have proven to be beneficial in prolonging the half-life of therapeutic protein drugs. It is expected that pegylation of the CDH11 polypeptides of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The CDH11 polypeptides containing these novel amino acids can then be pegylated at these chosen sites in the protein.

III. Methods of Treatment

The present invention provides particular novel therapeutic and diagnostic applications that employ CDH11 antagonists.

The terms “treat,” “treating,” and “treatment,” as used herein, refer to therapeutic or preventative measures described herein. The methods of “treatment” include administration of a CDH11 antagonist to a subject in order to prevent, cure, delay, reduce the severity of, or ameliorate one or more symptoms of a disease, condition or infection, in order to prolong the survival of a subject beyond that expected in the absence of such treatment.

The term “patient” includes human and other mammalian subjects that receive either prophylactic or therapeutic treatment.

As used herein, the term “subject” includes any human or non-human animal. For example, the methods and compositions of the present invention can be used to treat a subject having cancer. In a particular embodiment, the subject is a human. The term “non-human animal” includes all vertebrates, e.g., mammals and non-mammals, such as non-human primates, sheep, dog, cow, chickens, amphibians, reptiles, etc.

The term “sample” refers to tissue, body fluid, or a cell from a patient or a subject. Normally, the tissue or cell will be removed from the patient, but in vivo diagnosis is also contemplated. Other patient samples, include urine, tear drops, serum, cerebrospinal fluid, feces, sputum, cell extracts etc.

A. Indications

In one aspect, the methods of the present invention can be used to inhibit or prevent epithelial-mesenchymal transition (EMT) or endothelial-mesenchymal transition (EnMT). In a particular embodiment, the EMT or EnMT is associated with fibrosis.

Additionally, the methods of the present invention can be used to treat, prevent or reduce the severity of a wide variety of other conditions and diseases. Suitable diseases that can be treated and/or diagnosed using the CDH11 antagonists disclosed herein include, vascular fibrosis, vascular fibrosis associated with pulmonary hypertension, kidney fibrosis, liver fibrosis, skin fibrosis, lung fibrosis, fibrosis of the eyes (including systemic sclerosis (scleroderma)), fibrosis of the joints, fibrosis of the mesothelium and fibrosis of the gut (e.g., inflammatory bowel diseases). More specific types of fibrosis that can be treated include, but are not limited to Cystic fibrosis of the pancreas and lungs, Endomyocardial fibrosis, idiopathic myocardiopathy, Idiopathic pulmonary fibrosis of the lung, Diffuse parenchymal lung disease, Mediastinal fibrosis (i.e., characterized by invasive, calcified fibrosis centered on lymph nodes that block major vessels and airways), Myleofibrosis (i.e., a disorder of the bone marrow, in which the marrow is replaced by fibrous (scar) tissue), Retroperitoneal fibrosis (i.e., a disease featuring the proliferation of fibrous tissue in the retroperitoneum, the compartment of the body containing the kidneys, aorta, renal tract and various other structures), Progressive massive fibrosis (i.e., a disease that arises through the deposition of coal dust within the lung and then develops through the body's immunological reactions to the dust; also known as Complex Pneumoconiosis), Proliferative fibrosis, neoplastic fibrosis, fibrosis of the lungs caused by Tuberculosis (TB), fibrosis of the spleen caused by Sickle-cell anemia, and Rheumatoid arthritis.

The methods of the present invention can also be used to prevent or reduce the severity of chronic tissue rejection, i.e., such as the rejection of a transplanted or grafted tissue. Exemplary transplanted tissues include, but are not limited to bones, corneas, as well as major organs such as hearts, kidneys, livers, lungs, and pancreases.

In yet other embodiments, the CDH11 antagonists employed by the methods of the present invention may be used to treat immune disorders that include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia areata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cirrhosis; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; lupus nephritis; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Scleroderma, Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

B. Combination Therapies

CDH11 antagonists utilized in the methods of the present invention can be administered alone or in combination with other therapeutic agents. For example, the antagonists can be administered in combination with (i.e., together with or linked to (i.e., an immunoconjugate)) cytotoxins, other known therapeutic agents (i.e., immunosuppressive, chemotherapeutic agents, radiotoxic agents, and/or other therapeutic antibodies. The antagonist can also be administered separate from the agent. In the case of separate administration, the antagonist can be administered before, after or concurrently with the agent or can be co-administered with other known therapies, e.g., an anti-cancer therapy, e.g., radiation.

In one embodiment, the antagonist is linked to a second binding molecule, such as a second antibody (i.e., thereby forming a bispecific molecule) or other binding agent that binds to a different target or a different epitope on CDH11. Examples of additional therapeutic agents that can be used in combination therapy with the antagonists disclosed herein are described in greater detail above in the section on immunoconjugates.

C. Dosages/Amounts

The terms “effective amount” and “therapeutically effective amount” as used herein, refers to that amount of an antagonist, which is sufficient to effect treatment, prognosis or diagnosis of an infection or disease associated with increased expression of CDH11, as described herein, when administered to a subject. A therapeutically effective amount will vary depending upon the subject and the infection or disease condition being treated, the weight and age of the subject, the severity of the infection or disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody of the present invention. Dosage regimens may be adjusted to provide the optimum therapeutic response. An effective amount is also one in which any toxic or detrimental effects (i.e., side effects) of an antagonist are minimized and/or outweighed by the beneficial effects.

Actual dosage levels of the antagonists used in the methods of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antagonist employed, or the ester, salt or amide thereof, the route of administration, the time of administration, the rate of excretion of the particular antagonist being employed, the duration of the treatment, other drugs, compounds and/or materials used in combination with the particular antagonist employed, the age, sex, weight, condition, general health and prior medical history of the patient being treated, and like factors well known in the medical arts. A physician or veterinarian having ordinary skill in the art can readily determine and prescribe the effective amount of the antagonist required. For example, the physician or veterinarian could start doses of the antagonist at levels lower than that required in order to achieve the desired therapeutic effect and gradually increase the dosage until the desired effect is achieved. In general, a suitable daily dose of an antagonist will be that amount which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of an antagonist may be administered as two, three, four, five, six or more sub-doses administered separately at appropriate intervals throughout the day, optionally, in unit dosage forms. While it is possible for an antagonist of the present invention to be administered alone, it is preferable to administer the antagonist as a pharmaceutical formulation (composition).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the antagonists used in the methods of the present invention may be administered once or twice weekly by subcutaneous injection or once or twice monthly by subcutaneous injection.

It is especially advantageous to formulate parenteral antagonists in dosage unit form for ease of administration and uniformity of dosage. Dosage unit form as used herein refers to physically discrete units suited as unitary dosages for the subjects to be treated; each unit contains a predetermined quantity of active antagonist calculated to produce the desired therapeutic effect in association with the required pharmaceutical carrier. The specification for the dosage unit forms are dictated by and directly dependent on (a) the unique characteristics of the active antagonist and the particular therapeutic effect to be achieved, and (b) the limitations inherent in the art of compounding such an active antagonist for the treatment of sensitivity in individuals.

D. Methods of Administration and Formulations

To administer an antagonist used in the methods of the present invention by certain routes of administration, it may be necessary co-administer the antagonist with a material to prevent its inactivation. For example, the antagonist may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active antagonist, use thereof in a pharmaceutical compositions is contemplated. Supplementary active compounds can also be incorporated with the antagonist.

Therapeutic antagonists typically must be sterile and stable under the conditions of manufacture and storage. The antagonist can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including an agent that delays absorption, for example, monostearate salts and gelatin.

Sterile injectable solutions can be prepared by incorporating the active antagonist in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Therapeutic antagonists that can be used in the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the antagonist which produces a therapeutic effect. Generally, out of one hundred percent, this amount will range from about 0.001 percent to about ninety percent of active ingredient, preferably from about 0.005 percent to about 70 percent, most preferably from about 0.01 per cent to about 30 percent.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed along with the antagonists utilized in the methods of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The antagonists may also be administered with adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the antagonists used in the methods of the present invention are administered to humans and animals, they can be given alone or as a pharmaceutical antagonist containing, for example, 0.001 to 90% (more preferably, 0.005 to 70%, such as 0.01 to 30%) of active ingredient in combination with a pharmaceutically acceptable carrier.

The antagonists can be administered with medical devices known in the art. For example, in a preferred embodiment, an antagonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, antagonists can be formulated to ensure proper distribution in vivo. For example, the blood-brain barrier (BBB) excludes many highly hydrophilic compounds. To ensure that the antagonists cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

IV. Methods of Diagnosis

The present invention provides particular novel diagnostic applications to determine and predict whether a subject has or is at risk of developing a particular CDH11-associated condition. Methods for determining the prognosis of a subject diagnosed with a CDH11-associated condition are also provided. As used herein, the term “CDH11-associated condition” refers to any condition or disease associated with aberrant or elevated expression of CDH11, including any of the indications discussed above in the method of treatment section, such as EMT, EnMT, fibrosis (i.e., kidney fibrosis) or chronic allograft rejection.

In one embodiment, the present invention provides methods for assaying a sample from a subject for a particular biomarker, i.e., CDH11, wherein an aberrant or elevated level of CDH11 is indicative of a CDH11-associated condition, such as EMT, EnMT, fibrosis (i.e., kidney fibrosis) or chronic allograft rejection. In another embodiment, the present invention provides methods for diagnosing a CDH11-associated condition which comprises: (i) contacting a target sample with a reagent which reacts with CDH11; and detecting CDH11, wherein an elevated concentration of CDH11 relative to a normal control is indicative of a CDH11-associated condition.

Individuals suspected of having a CDH11-associated condition would benefit from having a diagnostic test aimed at detecting elevated or aberrant expression of CDH11 as early as possible so that disease progression can be retarded or even halted.

As used herein the term “biomarker” refers to any biologically-based marker of a condition. For example, a biomarker can be a biochemical feature or characteristic that can be used to objectively measure and evaluate normal biologic processes, pathogenic processes, or pharmacologic responses to a therapeutic intervention. In the present methods, CDH11 is a biomarker that can be used to assess whether a subject is at risk for developing a particular condition or disease. For example, in the present invention, elevated or aberrant expression of CDH11 can be correlated with a disease state (e.g., EMT, EnMT, fibrosis (i.e., kidney fibrosis) or chronic tissue rejection), as compared to a suitable control (e.g., the presence or level of the CDH11 in a normal or healthy sample). Suitable biologic samples also include blood samples, e.g., such as a plasma and/or serum, urine, stool, cerebrospinal (i.e. CSF) and spinal fluid, synovial fluid, conjunctival fluid, salivary fluid, lymph, bile, tears, and sweat), tissues and cells.

The “normal” level of a biomarker (i.e., CDH11) is the level of the biomarker in a subject or a sample from a subject (e.g., blood, e.g., serum or plasma, urine, stool, bile, tissues or cells, of a subject) who is not at risk of developing or who has not developed a disease or condition associated with CDH11 expression (i.e., EMT, EnMT, fibrosis (i.e., kidney fibrosis) or chronic tissue rejection) (e.g., sample from a subject not having the CDH11 associated disease). A “control” subject typically has normal levels of the biomarker, i.e., CDH11.

“An aberrant level” of a biomarker is any level of a biomarker that differs from the normal level of, e.g., significantly higher or elevated levels, or significantly lower or depressed levels of a biomarker.

A “higher level,” “elevated level,” or “increased level” of a biomarker refers to a level that is elevated relative to a suitable control. Preferably, the differential from the suitable control, if any, is greater than the standard error of the assay employed to assess the level. Moreover, the elevated level is preferably at least twice, and more preferably three, four, five or ten times the level of the biomarker in a suitable control (e.g., sample from a subject not having the biomarker associated disease, or the average level of the biomarker in several control samples or other suitable benchmark).

A “depressed level,” “lower level” or “decreased level” of a biomarker refers to a level that is depressed relative to a suitable control. Preferably, the differential from the suitable control, if any, is greater than the standard error of the assay employed to assess the level. The depressed level preferably is at least twice, and more preferably three, four, five or ten times lower than the level of the suitable control (e.g., level in a healthy subject not having the biomarker associated disease or the average level of the biomarker in several control samples or other suitable benchmark).

A. Diagnostic Assays

The presence, absence, and/or level of CDH11 may be assessed by any of a wide variety of well known methods for detecting a molecule or protein. Non-limiting examples of such methods include immunological methods for detection of proteins, protein purification methods, protein function or activity assays, nucleic acid hybridization methods, nucleic acid reverse transcription methods, and nucleic acid amplification methods, ELISA, immunoblotting, Western blotting, Northern blotting, Southern blotting and the like.

In one embodiment, the presence, absence, and/or level of CDH11 is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically to the biomarker, i.e., CDH11, such as the protein encoded by the open reading frame corresponding to the biomarker or such a protein which has undergone all or a portion of its normal post-translational modification. The term “labeled”, with regard to the antibody, is intended to encompass direct labeling of the antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody, such that it can be detected with fluorescently labeled streptavidin. In another embodiment, the presence, absence, and/or level of CDH11 is assessed using a nucleic acid.

The detection methods of the invention can be used to detect CDH11, for example, in a biological sample in vitro as well as in vivo. For example, in vitro techniques for detection of mRNA include Northern hybridizations, in situ hybridizations and QPCR. In vitro techniques for detection of CDH11 include, for example, enzyme linked immunosorbent assays (ELISAs), Western blots, immunoprecipitations and immunofluorescence. In vitro techniques for detection of CDH11 DNA include, for example, Southern hybridizations. Furthermore, in vivo techniques for detection of CDH11 include introducing into a subject a labeled antibody directed against CDH11. As discussed above, the antibody can be labeled with a radioactive biomarker whose presence and location in a subject can be detected by standard imaging techniques.

The present invention is further illustrated by the following examples which should not be construed as further limiting. The contents of Sequence Listing, figures and all references, patents and published patent applications cited throughout this application are expressly incorporated herein by reference.

### Examples

### Materials and Methods

### I. Data Collection

A. Human Samples

The biopsy samples used in this study were obtained from two external sources. The first dataset (referred to herein as the “Hannover dataset”) consists of kidney protocol biopsies which were available through a collaboration with the Transplant Center at the Medical School of Hannover. The study focused on month three biopsies from twenty renal allograft recipients with functional grafts and normal postoperative clinical and histologic parameters. Three months later, at the month six biopsy, eight of these patients had diagnosed chronic allograft rejection (CR) (progressor), while twelve of the patients maintained stable grafts (non-progressor).

The second collaboration was done in conjunction with the Hospital Tenon Paris, which gathered a total of 48 diagnostic biopsies, including 19 controls, 7 grade I, 7 grade II and 8 grade III biopsies. Additionally, 7 acute rejection (AR) biopsies having no signs of fibrosis, but associated with a severe allograft immune response were included. This dataset is referred to herein as the “Paris dataset.”

To further validate the study, a relevant microarray experiment from Gene Expression Omnibus (GEO) (#GSE6004) was downloaded and the gene expression profiles of microscopically dissected intratumoral samples from central and invasive regions of seven widely invasive papillary thyroid carcinomas (PTCs) and normal thyroid tissue by HG_U133_Plus2 Affy chip 10 were analyzed. Total RNA was obtained from seven central and invasion regions, as well as from four of seven normal tissues. In addition, the comparison of central versus normal tissues was compared to nine paired central and normal samples from The Ohio State University tumor bank simultaneously analyzed using the same methods.

B. Nonhuman Primate (NHP) Samples

Cynomolgus monkey (Macaca fascicularis) kidney allografts and controls were collected at necropsy from a life-supporting acute rejection model and a recently published chronic allograft vasculopathy study 11 with histopathological assessment of rejection. Total RNA from the renal cortex was extracted and processed without amplification using Affymetrix standard protocol and HG-U133A genechips.

### II. Microarrays

For each human biopsy, 50 ng of total RNA was subjected to the validated Affymetrix 2-cycle cDNA amplification, fluorescent labeling, and hybridization to the Human Genome U133 Plus 2.0 ArrayHG-U133 Plus2 human genome array hybridization (Affymetrix, Santa Clara, Calif.). A sample of 1 μs of total RNA was processed, labeled, and hybridized the samples to the Human Genome U133 Plus 2.0 ArrayAffymetrix HG-U133_plus2 Genechip genechip (containing ˜54,625 probe sets for >47,000 different human transcripts) (Affymetrix, Santa Clara, Calif.). Total RNA from renal cortex of NHP samples was extracted and processed without amplification using Affymetrix standard protocol and HG-U133A genechips.

III. Statistical analysis

A single weighted mean expression level for each gene along with a p-value indicating reliable transcript detection was derived using Microarray Suite 5.0 software (MASS, Affymetrix). Data were scaled from each array (target intensity of 150). For further analysis, the cell intensity (CEL) files were subjected to the Robust Multichip Analysis (RMA) normalization. Several quality control measures on each array were assessed, including review of the scanned image for significant artifacts, background and noise measurements that differ significantly from other chips, average of present and absent calls. Arrays failing two out of the three following criteria were excluded from the study: (1) the 3′ to 5′ ratio of the intensities for glyceraldehyde-3-phosphate dehydrogenase (ratio ≦4), (2) average of calls (≧40% of present calls), and (3) scaling factor (or ≧2 or ≦0.5).

Analysis of the raw data was performed using GeneSpring GX7.3 (Agilent Technologies). Genes differentially expressed among different sample classes were identified using a one-way ANOVA (p ≦0.05), with or without a false discovery rate ≦5% (Benjamin and Hochberg FDR) and additional cutoff based on 2-fold change between groups.

An implementation of the Gene Set Enrichment Analysis (GSEA), using publicly available set of genes (KEGG, Celera public, and Mootha 12) was applied. GSEA results in a list of gene sets associated with corresponding p-values. A small p-value indicates that the gene set is significantly enriched at either the top or the bottom of the list of expression ratios.

### Example 1

### Identification of the EMT Gene Set

In this example, genes were identified and selected that are significantly over-expressed in early and late chronic rejection. The overlap of these two comparisons generated a list of 287 probesets that were found to have significantly changed in progressor patients at three months post-transplantation (Hannover dataset) and in patients with grade III chronic allograft rejection (Paris dataset), as compared to normal, stable patients.

Next, genes having an expression profile highly correlating with EMT markers in both datasets were selected. Snail2 was chosen as bait for the correlation analysis for the following reasons. First, Snail2 is known for its ability to trigger EMT, thereby converting epithelial cells into mesenchymal cells with migratory properties, and has been shown to induce renal fibrosis in transgenic mice and pathological models. Snail2 expression is also detected in human fibrotic kidneys (Boutet A et al, EMBO Journal, 2006). Additionally, Snail2 is an EMT marker that is highly up-regulated in both early and late chronic rejection samples.

A Pearson correlation (r>0.8) was then applied across each entire dataset. The overlap of the two correlation gene lists generated a list of 184 probesets, which consistently correlated with Snail2 in both the Paris and Hannover datasets.

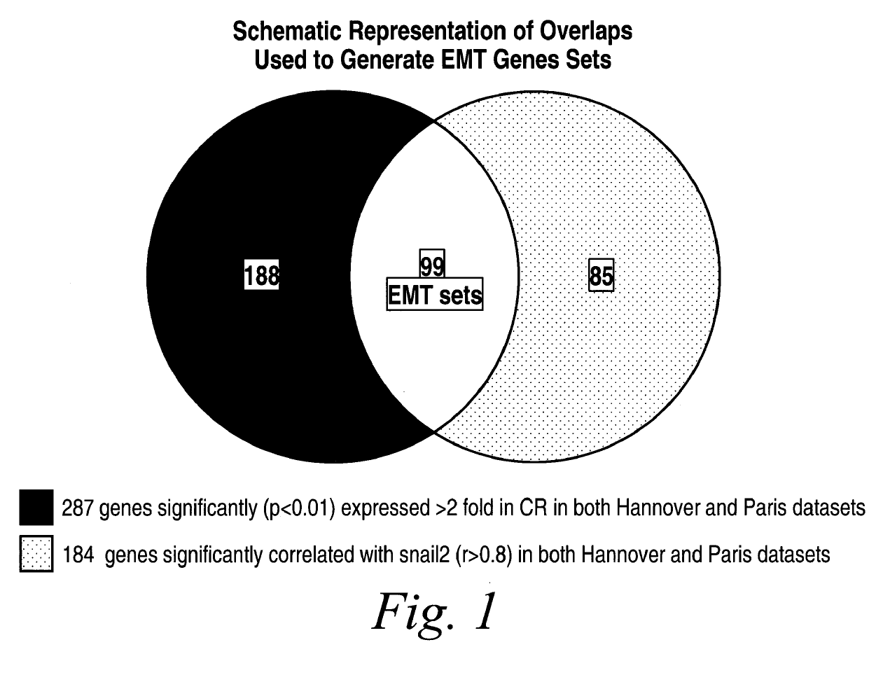

Finally, a first list of genes that are significantly up-regulated during chronic allograft rejection (287 probesets) was overlapped with a second list of genes that correlated with Snail2 (184 probesets). FIG. 1 is a schematic representation of the overlap used to generate the EMT gene sets. Based on this overlap, a final gene list of 99 probesets was identified, including cadherin 11, as set forth in Table 1.

TABLE 1

| Ids | Entrezgene | Description |
|---|---|---|
| 207173_x_at | 1009 | cadherin 11, type 2, OB-cadherin |
| | | (osteoblast) (CDH11) |

### Example 2

### Validation of EMT Gene Set

A. GSEA Analysis of Papillary Thyroid Carcinoma (PTC) Invasion Gene Expression

In order to validate the relevance of the identified gene set as a pathogenic molecular signature for EMT, a gene set enrichment method (GSEA) was applied to this “EMT” gene set on relevant microarray datasets. After mining of the GEO database, a particular dataset by Vasko et al., which describes a transcriptomics analysis of papillary thyroid carcinoma tumor associated with EMT, in vivo, was identified (see Vasko, V. et al. Proceedings of the National Academy of Sciences 104, 2803-2808 (2007)). Vasko et al. compare the central part with the invasive part of the same tumor and state that the most significant genes changes were involved in cell-cell adhesion and communication, consistent with epithelial-to-mesenchymal transition (EMT).

To confirm that aggressive papillary thyroid carcinomas are characterized by EMT, 34 additional papillary thyroid carcinomas were examined for expression of vimentin, which is a hallmark of EMT. This analysis revealed that over-expression of vimentin was associated with papillary thyroid carcinoma invasion and nodal metastasis. Additionally, functional in vitro studies demonstrated that vimentin was required both for the development and maintenance of a mesenchymal morphology and invasiveness in thyroid cancer cells. Based on these studies it is clear that EMT is common in papillary thyroid carcinoma invasion and that vimentin regulates thyroid cancer EMT in vivo.

Using this dataset, a GSEA comparison between the central and invasive part of the papillary thyroid carcinoma tumor associated with EMT features was performed. Consistent with published results, very few pathways were found to be differentially expressed between the two different parts (i.e., central and invasive) of the same tumor, as shown in Table 2.

TABLE 2

| Pathway | Source | Gene | Q-Value | Direction |
|---|---|---|---|---|
| EMT | Novartis | 99 | 1.01E−05 | Top |
| Ataxia Telangiectasia | Metacore | 21 | 0.00129 | Top |
| DNA Repair | Celera | 226 | 0.0036 | Top |
| Pyrimidine Metabolism | Kegg | 119 | 0.00532 | Top |
| Multiple Sclerosis | Metacore | 171 | 1.57E−07 | Bottom |
| Natural Killer Cell | Celera | 104 | 1.44E−06 | Bottom |
| Mediated Immunity | | | | |
| Interleukin Signaling | Pathart | 221 | 1.55E−06 | Bottom |
| Pathway | | | | |
| TNF Signaling Pathway | Pathart | 154 | 6.49E−06 | Bottom |
| MHCII-Mediated | Celera | 44 | 8.56E−06 | Bottom |
| Immunity | | | | |
| Cytokines | BC | 245 | 1.23E−05 | Bottom |

However, a couple of pathways associated with inflammation and immune response were found to be significantly down-regulated in the invasion part of the tumor and two pathways were found to be significantly up-regulated. Remarkably, the most significant pathway identified in the invasive part of the tumor is the “EMT” gene set, defined in the previous example. This result suggests that the expression profile of the EMT gene set may be relevant to monitor pathogenic EMT process occurring in vivo in humans.

B. GSEA Analysis of Early and Late Chronic Allograft Rejection

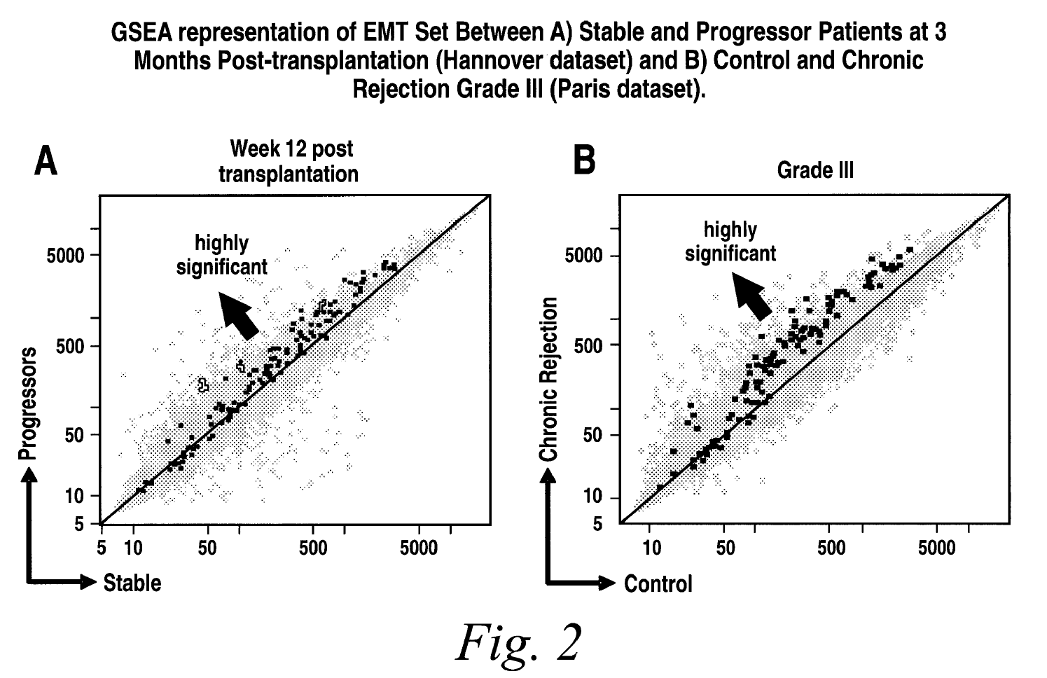

In order to evaluate if the EMT gene signature is regulated across the different stages of chronic allograft rejection and interstitial fibrosis progression, the GSEA method was applied to both progressor and non-progressor patients from the Hannover data set, as well as to control and chronic allograft rejection grade III patient samples (FIG. 2). As shown in FIG. 2B, the EMT set is significantly up-regulated among more than a thousand pathways in grade III patients when compared to control patients. Surprisingly, the same gene set is one of the most significantly regulated pathway in the progressor patient group at week 12 post-transplantation (FIG. 2B).

### Example 3

### Identification of CDH11 as Key Target to Block EMT and Interstitial Fibrosis Progression

Among the genes identified in the EMT set, several are well known EMT markers, such as vimentin or S100A4. However, others are newly identified EMT-associated genes, including CDH11, which may be considered as putative therapeutic targets for fibrosis. Accordingly, to confirm whether CDH11 is actually involved in pathogenic EMT during kidney fibrosis progression, the expression profile of CDH11 at both the mRNA and protein level was analyzed.

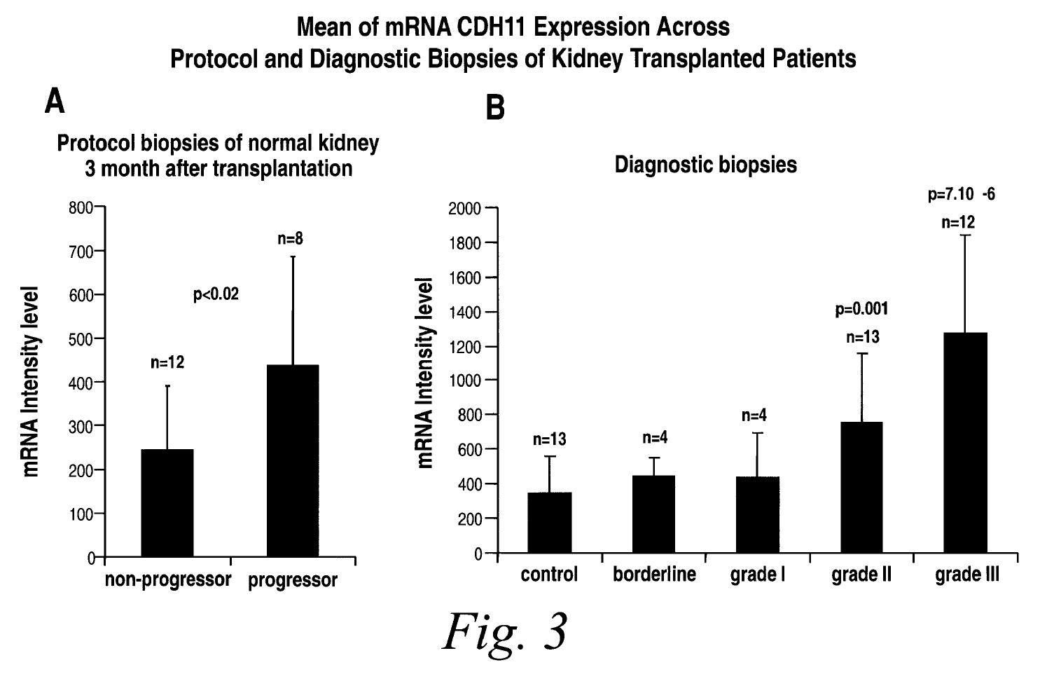

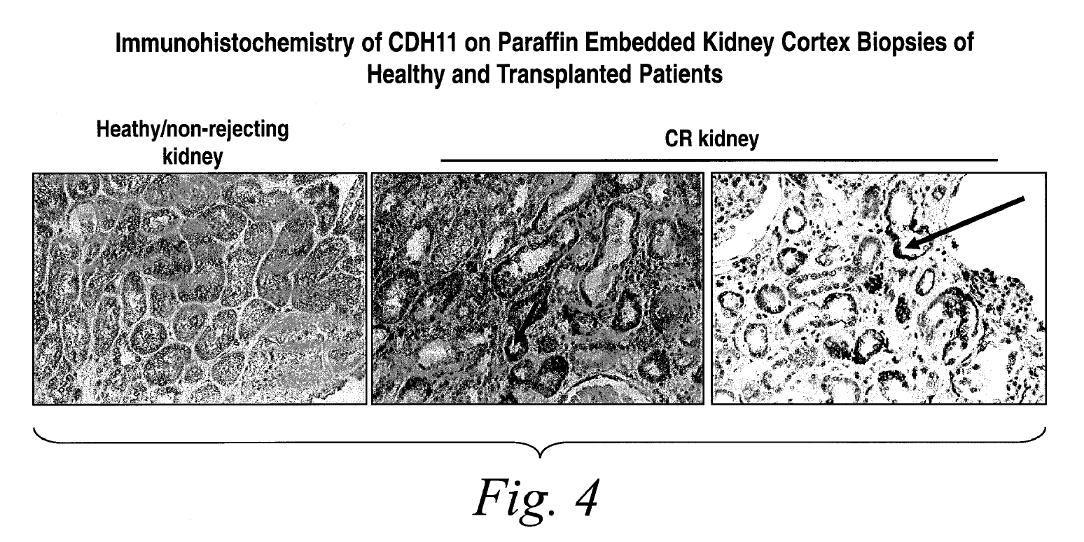

As shown in FIG. 3A, CDH11 expression is consistently up-regulated in the early phase of kidney fibrosis, as well as during the later states of the disease (FIG. 3B). In addition, CDH11 was consistently found co-expressed with Snail2, the EMT positive marker used in this study. One striking observation is that CDH11 is similarly co-expressed with Snail2 across more than 80 biopsies from the Hannover dataset (Table 3) as well as across more than 80 biopsies of the Paris dataset (Table 4).

TABLE 3
Gene Highly Correlated with Snail2 in Hannover Dataset

| IDs | r value | FC* | Symbols |
|---|---|---|---|
| 213139_at | 1 | 1.245919 | SNAIL2 |
| 207173_x_at | 0.757 | 1.792251 | CDH11 |

*FC Based on Non-Progressor vs. Progressor Comparison at Week 12 Post Transplantation

TABLE 4
Genes Highly Correlated with Snail2 in Tenon Dataset

| IDs | r value | FC* | Symbol |
|---|---|---|---|
| 213139_at | 1 | 3.057614 | SNAI2 |
| 202995_s_at | 0.824 | 3.560018 | FBLN1 |
| 211896_s_at | 0.823 | 2.666715 | DCN |
| 207173_x_at | 0.791 | 3.860664 | CDH11 |
| 203222_s_at | 0.777 | 2.271124 | TLE1 |
| 203903_s_at | 0.776 | 2.49614 | HEPH |
| 204396_s_at | 0.77 | 3.296966 | GPRK5 |
| 201438_at | 0.766 | 5.642283 | COL6A3 |
| 202202_s_at | 0.763 | 4.347918 | LAMA4 |
| 202766_s_at | 0.755 | 6.86041 | FBN1 |
| 203851_at | 0.744 | 5.601282 | IGFBP6 |
| 201761_at | 0.743 | 4.101709 | MTHFD2 |

*FC Based on Control vs Grade III Comparison

Considering that co-expression analysis may traduce similar transcriptional regulation, this result suggests that CDH11 is regulated in the same way as Snail2 and contribute to the EMT process.

Because this result comes from a global gene expression of the entire kidney cortex, it is crucial to understand in which cells types CDH11 protein is expressed (e.g., tubules, glomeruli, mesenglial cells, vascular and endothelial cells, etc.). EMT is classically detected in vivo by immunohistochemistry (IHC) staining of mesenchymal markers, such as vimentin or FSP1 (S100A4) (See Kalluri, R. & Neilson, E. G., J. Clin. Invest. 112, 1776-1784 (2003)). In the normal kidney, epithelial tubular cells of the cortex display a negative staining for vimentin. However, when a transient EMT process begins, several normal and early atrophic tubules start to express vimentin and lose E-cadherin staining. This peculiar staining is observed in 40% of the kidney (see Hertig, A. et al, American Journal of Transplantation 6, 2937-2946 (2006)).

Therefore, in order to test if CDH11 is expressed with EMT markers, IHC was performed on healthy, non-rejecting kidney biopsies and chronic allograft rejection kidney biopsies using a poly-rabbit antibody against human CDH11. As expected, anti-CDH11 shows no or very low staining in normal or non-rejecting kidneys, but display a strong tubular staining in chronic allograft rejection kidneys (FIG. 4). This staining is typical of classical EMT staining, such as vimentin, and further confirms the potential role of CDH11 in EMT and kidney fibrosis in human.

### Example 4

### Identification of CDH11 as Key Target to Block EnMT and Vascular Remodeling

Non-human primate (NHP) models of acute and chronic kidney transplant rejection provides a useful tool to study and understand the arterial remodeling observed in patients with chronic allograft rejection (see Wieczorek, G. et al. American Journal of Transplantation 6, 1285-1296 (2006)). In this model, animals developed chronic allograft rejection and lost grafts within 65 days (median). As compared to acute rejection, the arterial intimal changes showed less macrophages and T lymphocytes, but increased number of myofibroblasts, abundant fibronectin/collagen IV and scar collagens I/III. Interstitial fibrosis and tubular atrophy are not very prominent features of this experimental model, presumably due to the relatively short duration of impaired blood flow and ischemia-induced atrophy/fibrosis secondary to arterial stenosis.

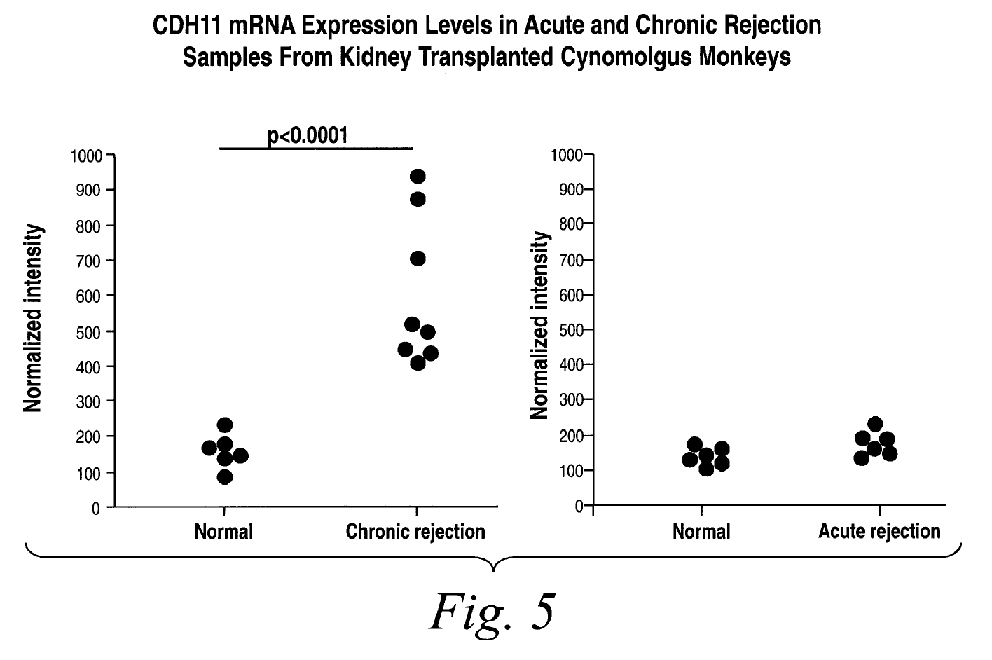

In order to get better insight into this process, a full transcriptomics experiment was conducted on kidney biopsies from monkeys diagnosed as normal or displaying clinical signs of acute and chronic rejection. After classical statistical filtering, CDH11 was identified as one gene specifically over-expressed in biopsies with vascular remodeling and an increased number of myofibroblasts was observed as compared with control and samples with acute rejection (FIG. 5). This data suggests that, in addition to interstitial fibrosis, CDH11 might also play a role in vascular remodeling and intimal hyperplasia.

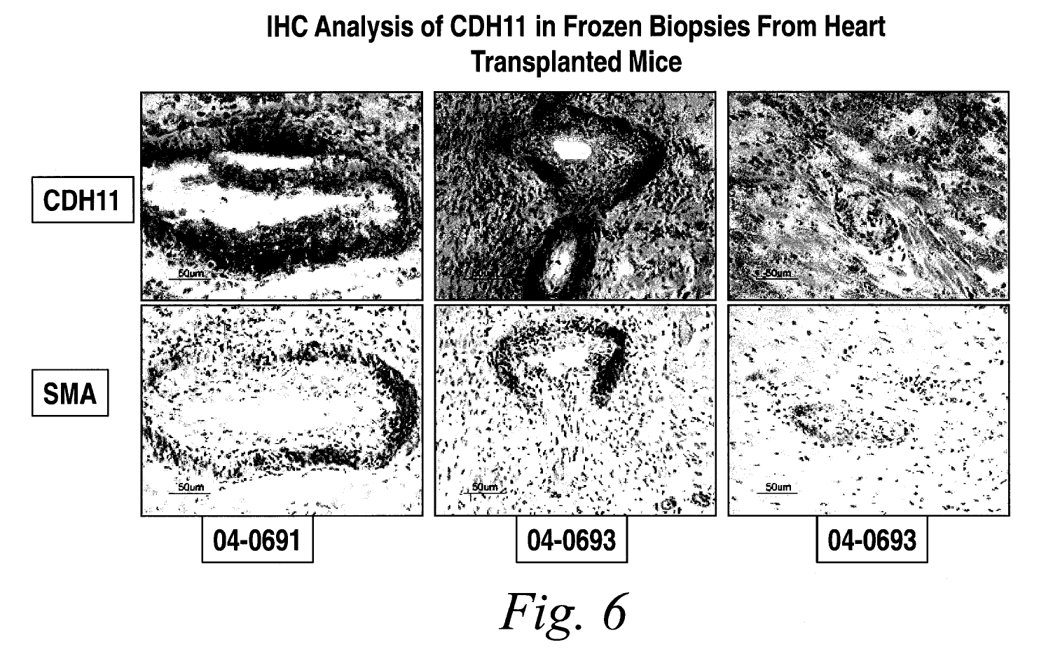

To further assess the potential role of CDH11 in vascular remodeling, an immunohistochemistry analysis was performed using additional animal models. Because of the lack of satisfactory cross-reactive specific antibodies for cynomolgus monkeys, CDH11 expression was tested in a mouse model of heart chronic rejection associated with vascular remodeling and intimal hyperplasia. FIG. 6 shows that the presence of CDH11 signal was detected in some intramural coronary arteries with intima thickening. In the affected vessels, media seemed to be strongly positive and intima showed only weak positivity. In addition, the signal appears co-expressed with alpha SMA staining, a specific marker for smooth muscle cells and myofibroblasts (FIG. 6). Importantly, CDH-11 staining cannot be detected in healthy vessels.

### Example 5

### Demonstration of Cadherin-11 as an EMT Marker in Mouse Model of Kidney Fibrosis (UUO)

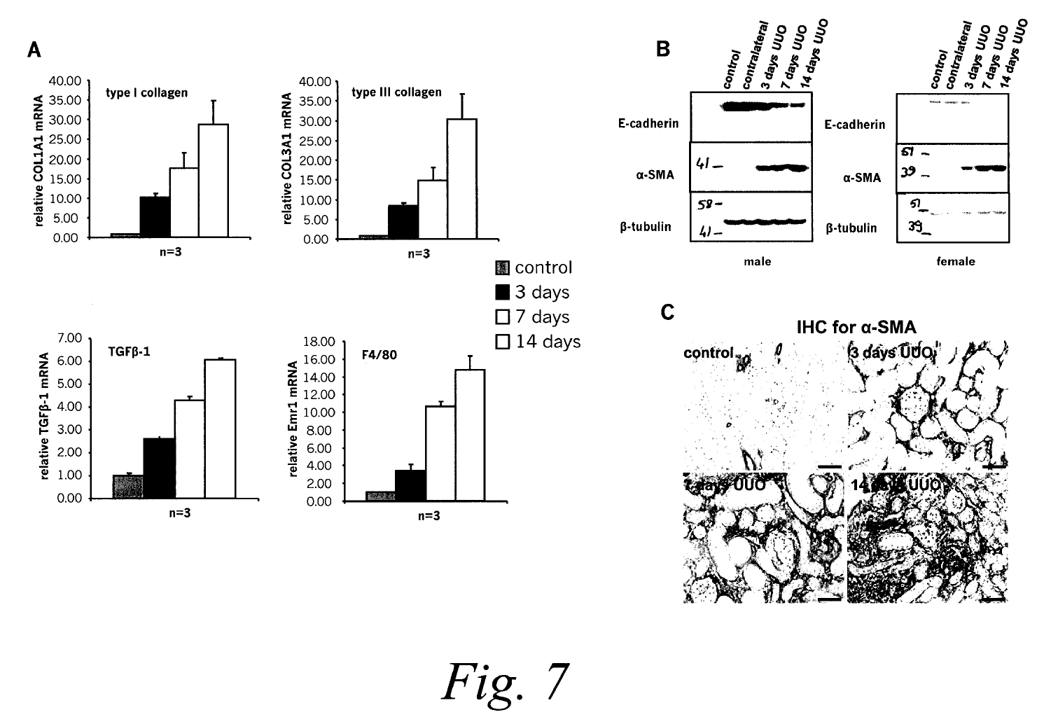

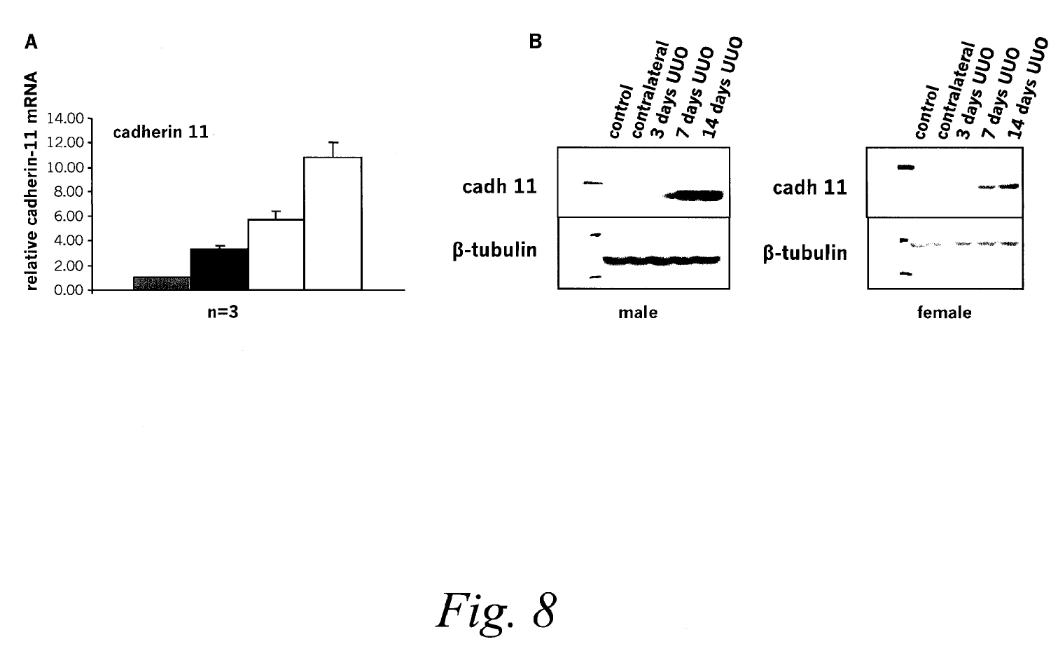

Unilateral ureteral obstruction (UUO) mouse models are well established and documented, and broadly used to study fibrosis, e.g., progressive interstitial fibrosis and kidney fibrosis, and to evaluate potential therapeutic approaches. The ureteral obstruction surgery in these models induces a rapid development of tubulointerstitial fibrosis (1-2 weeks) which is highly reproducible. UUO is known to induce a strong up regulation of collagens, TGFβ, α-SMA, and a significant diminution of E-cadherin. As shown at least in FIG. 7, UUO induces a strong up regulation of CDH11 in a time dependent manner.

### Equivalents

Those skilled in the art will recognize, or be able to ascertain using no more than routine experimentation, many equivalents of the specific embodiments of the invention described herein. Such equivalents are intended to be encompassed by the following claims. Any combination of the embodiments disclosed in the dependent claims are contemplated to be within the scope of the invention.

### INCORPORATION BY REFERENCE

All publications, patents, and pending patent applications referred to herein are hereby incorporated by reference in their entirety.

1. A method of inhibiting or preventing epithelial-mesenchymal transition (EMT) or endothelial-mesenchymal transition (EnMT) in a subject comprising administering to the subject a therapeutically effective amount of a CDH11 antagonist, thereby inhibiting or preventing epithelial-mesenchymal transition (EMT) or endothelial-mesenchymal transition (EnMT).

2. The method of claim 1, wherein the EMT or EnMT is associated with fibrosis.

3. A method of treating fibrosis in a subject, comprising administering to the subject a therapeutically effective amount of CDH11 antagonist.

4. The method of claim 3, wherein the fibrosis is selected from the group consisting of vascular remodeling, kidney fibrosis, liver fibrosis, skin fibrosis, lung fibrosis, fibrosis of the joint, fibrosis of the mesothelium and fibrosis of the gut.

5. The method of claim 4, wherein the vascular fibrosis is associated with pulmonary hypertension.

6. A method of preventing or reducing the severity of chronic tissue rejection in a subject, comprising administering to the subject a therapeutically effective amount of a CDH11 antagonist.

7. The method of claim 6, wherein the tissue is a transplanted tissue or a grafted tissue.

8. A method of treating kidney fibrosis in a subject, comprising administering to the subject a therapeutically effective amount of a CDH11 antagonist, wherein the CDH11 antagonist is selected from the group consisting of an antibody, a small molecule, a nucleic acid, a fusion protein, and a CDH11-derived peptidic compound.

9-10. (canceled)

11. The method of claim 1, wherein the antagonist is selected from the group consisting of an antibody, a small molecule, a nucleic acid, a fusion protein, and a CDH11-derived peptidic compound.

12. The method of claim 11, wherein the antibody is selected from the group consisting of a murine antibody, a human antibody, a humanized antibody, a bispecific antibody and a chimeric antibody.

13. The method of claim 11, wherein the antibody is selected from the group consisting of a Fab, Fab′2, ScFv, SMTP, affibody, avimer, nanobody, and a domain antibody.

14. The method of claim 11, wherein the antibody is administered in combination with a second therapeutic agent.

15. The method of claim 14, wherein the second therapeutic agent is selected from the group consisting of a second antibody, an immunosuppressant, and a chemotherapeutic.

16. The method of claim 11, wherein the nucleic acid is an antisense molecule selected from the group consisting of an RNA interfering agent and a ribozyme.

17. The method of claim 1, wherein the antagonist is an immunoconjugate comprising an antibody linked to a therapeutic agent.

18. The method of claim 17, wherein the therapeutic agent is selected from the group consisting of a cytotoxic agent, an immunosuppressive agent, and a chemotherapeutic agent

19. A method of assessing whether a subject has or is at risk of developing a CDH11-associated condition; comprising assaying a sample from a subject for CDH11, wherein an aberrant or elevated level of CDH11 indicates that the subject has or is at risk of developing a CDH11-associated condition.

20. A method for diagnosing a CDH11-associated condition comprising: (i) contacting a target sample with a reagent which reacts with CDH11; and (ii) detecting CDH11, wherein an elevated concentration of CDH11 relative to a normal control is indicative of a CDH11-associated condition.

21. A method of determining the prognosis of a subject diagnosed with a CDH11-associated condition comprising assaying at least two samples from the subject which have been collected over time, comparing the levels of CDH11 in each sample, and determining if the level of CDH11 has increased or decreased over time, wherein an increase in CDH11 is indicative of an increase in severity of the condition and a decrease in CDH11 is indicative of a decrease in severity of the condition.

22. The method of claim 20, wherein the CDH11-associated condition is selected from the group consisting of EMT, EnMT, fibrosis, and chronic tissue rejection.

23. The method of claim 20, wherein the reagent is an antibody or a nucleic acid.

24. The method of claim 23, wherein the reagent is detectably labeled.

25. The method of claim 24, wherein the label is selected from the group consisting of a radioisotope, a bioluminescent compound, a chemiluminescent compound, a fluorescent compound, a metal chelate, or an enzyme.

26. The method of claim 21, wherein the subject has been treated with a CDH11 antagonist.

27. The method of claim 26, wherein the CDH11 antagonist is selected from the group consisting of an antibody, a small molecule, a nucleic acid, a fusion protein, and a CDH11-derived peptidic compound.


SEQUENCE LISTING (1 sequence)

SEQ ID NO: 1 - protein, 796 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQSVYQMSVSEAAVPGEEVGRVKAKDPDIGENGLVTYNIVDGDGMESFEITTDYETQEGVIKLKKPVDFETKRAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAKVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLVIVVLFVTLRRQKKEPLIVFEEEDVRENIITYDDEGGGEEDTEAFDIATLQNPDGINGFIPRKDIKPEYQYMPRPGLRPAPNSVDVDDFINTRIQEADNDPTAPPYDSIQIYGYEGRGSVAGSLSSLESATTDSDLDYDYLQNWGPRFKKLADLYGSKDTFDDDS